(19) United States
(12) Patent Application Publication
(10) Pub. No.: US 2005/0040042 A1
(43) Pub. Date: Feb. 24, 2005

(54) Method and device for electronic control of the spatial location of charged molecules

(76) Inventors: Jae-young Yun, Seoul (KR); Hong-gil Nam, Seoul (KR); Seung-seob Lee, Pohang-city (KR); Sang-jun Moon, Pohang-city (KR)

Correspondence Address: LEYDIG VOIT & MAYER, LTD, 700 THIRTEENTH ST. NW, SUITE 300, WASHINGTON, DC, 20005-3960, US

(21) Appl. No.: 10/344,767

(22) PCT Filed: Jun. 14, 2001
(86) PCT No.: PCT/KR01/01019

(51) Int. Cl. G01L 1/20; C07K 1/26

(52) U.S. Cl. 204/450; 204/600

(57) ABSTRACT

A method and device for electronically controlling the spatial location of charged molecules is described. The method disclosed pertains to controlling the spatial location of charged molecules in a reaction unit wherein a plurality of electrodes are placed, wherein the geometry of electrode placement is closed system such as circular, rectangular or diamond-shape. This control extends to the selective isolation of individual species of charged molecules as well as their transport. The invention is useful in molecular biological reactions, such as nucleic acid hybridizations, nucleic acid amplification, sample preparation, antibody/antigen reactions, clinical diagnostics, and biopolymer synthesis.

### BACKGROUND OF THE INVENTION

1. Field of the Invention

This invention pertains to the design and uses of an addressable microelectronic system that can actively control the spatial location of charged molecules in microscopic formats. Said control is based on electrophoretic forces and allows for the selective isolation and transport of specific molecular species. One embodiment of this method is in the control of biochemical reactions. In particular, these reactions include molecular biological reactions, such as nucleic acid hybridizations, nucleic acid amplification, sample preparation, antibody/antigen reactions, clinical diagnostics, and biopolymer synthesis.

2. Description of the Related Art

Electrophoresis is the movement of charged molecules in electric fields, a method that has been used as an analytical technique to separate and identify charged particles, ions, or molecules.

The most often studied molecules are biological molecules such as proteins and DNA fragments, which are usually polyelectrolytes. Separation is the most frequent use of electrophoresis in life science. Molecules are separated by their different charge and frictional resistance characteristics. The electrophoretic mobility (U) of a charged molecule in an electric field (E) is defined as the ratio of its center of mass velocity (Vg) to the external applied electric field. (Vg=UE). The more charged and streamlined the molecules, the faster their movement.

When a mixture containing several molecular species is introduced into an electrophoretic separation medium and an electric field is applied, the different charged components migrate at various speeds in the system leading to the resolution of the mixture. Bands appear, depending on the nobilities of the components. The exact location (thus time of emergence of the components at the end of the medium opposite to the point of introduction) depends on the interaction of the polyelectrolytes with the surrounding medium, via the influence of pH, ionic strength, ion type and whether the medium is a buffered solution of ions, polymeric solution, or gel such as a cross-linked gel. Cross-linked gels and polymeric solutions can affect separation by size or sieving. Hence, electrophoresis can be classified into two basic types including (1) free solution and (2) gel electrophoresis. The most frequently used gel media are based on polyacrylamide (known as PAGE) and agarose gels.

The combination of free solution and gel electrophoretic separation experiments gives a plethora of information, such as the number and relative amounts of the components in a mixture. When the components are specifically identified, e.g., by antigen-antibody binding, unequivocal identification of the presence of the given component is afforded. As a consequence, electrophoresis has become the cornerstone of macromolecular analysis in biotechnology.

U.S. Pat. No. 5,126,022 (Soane, et al., assigned to Soane Technologies, Inc.) discloses a typical method and device for moving charged molecules. This method requires a plurality of different electrical fields being applied to medium such as buffer solution in order to move molecules within the medium in a precise manner. This technology may be used to move together, and separate from each other, charged particles in order to carry out complex reaction and/or separation schemes.

WO 99/62622 (Arnold, William, and Michael, assigned to Industrial Research Limited) discloses a method for concentrating and positioning particles in the medium by applying voltage to electrode array. In this method, the electrode array comprises two comb-like rows of spaced interdigitated finger-like electrode elements to levitate the particles within the medium. Arnold, et al. have not demonstrated this method provide a way to conduct biological reactions by concentrating the particles into a specific zone. This method provides a means by which to position particles at specific zones in a medium for the purpose of separation or identification of specific particles from the remainder of the buffer constituents.

U.S. Pat. No. 6,017,696 (Michael J. Heller, assigned to Nanogen, Inc.) discloses a method for electronic stringency control for molecular biological analysis and diagnostics. In this method, molecules are transported into a micro-location by electrophoretic force so that the speed of biological analysis can be considerably enhanced. This method also takes advantage of the electric field for stringency control so that various biological analyses including DNA hybridization can be carried out quickly.

However, in this method, negatively and positively charged molecules cannot be manipulated at the same time. It requires the polarity of electrodes to alternate from negative to positive in order to manipulate charged molecules. For example, polymerase chain reaction (PCR) requires the interaction between negatively charged DNA molecules and positively charged ions such as K⁺, Mg²⁺ and slightly positively charged Taq polymerase. For PCR, the Nanogen method cannot manipulate all of the reactants necessary for the reaction at the same time. This is because DNA molecules should be transported into a reaction unit and be bound to the electrodes, and then the polarity of electrode must be changed to wash out unbound DNA molecules. The polarity of the electrode must be changed again to transport Taq polymerase into the reaction unit. This process is time-consuming and repeating it for DNA extension is not practical.

This feature limits the design of a device for biological analysis because of the complex process described above. The functionality is also more limited in regards to use in new biological analysis or new diagnostic techniques in which stringency control is manipulated between negatively charged molecules and positively charged molecules.

Methods to separate and store ions by applying an electric field to a trap volume are well known. U.S. Pat. No. 6,124,592 (Spangler; Glenn E., assigned to Nikaido, et al.) discloses a method to separate and store ions by exploiting mobility characteristics of the ions. In this method, the ions are separated according to their mobility characteristic by applying an electric field to the trap volume. The ions then migrate to equilibrium positions in the trap volume due to a difference in motilities and to changes in the electric field. The ions can be positively or negatively charged, and the motion of the ions depends on the direction and magnitude of the applied electric field.

In this method, the ions cannot be localized to speed up desirable reactions such as hybridization. A sample that is ionized in the trap volume by applying the electric field cannot be addressed into a specific test site. So this method can be used in ion mobility spectrometry (IMS) only.

WO 97/34689 (Pething, et al., assigned to University College of North Wales) discloses an apparatus for carrying out chemical, physical or physico-chemical reactions between particles suspended in a liquid medium. In this method, the particles are moved using dielectrophoresis or traveling wave dielectrophoresis.

There are few inventions related to the use of electrophoresis in biochips, such as a lab-on-a-chip. One of the reasons to invent biochips using electrophoresis is the fact that many biological reactions require the manipulation of two oppositely charged molecules. Conventional inventions have difficulty in manipulating two oppositely charged molecules for biological reactions.

This difficulty has been solved through the geometry of electrode placement and the pattern of applied voltage in a reaction unit. We invented a reaction unit wherein a single central electrode is surrounded by a plurality of outer electrodes to manipulate two oppositely charged molecules at the same time.

### SUMMARY OF THE INVENTION

The device and the related methodologies of this invention allow molecular biology and diagnostic reactions to be carried out under complete electronic control. The methods and device are described for controlling the spatial location of charged molecules by applying an electric field to a reaction unit wherein multiple electrodes are placed. The control of spatial location of charged molecules can position the molecules necessary for biological analysis into a limited zone so that the speed of analysis can be enhanced.

According to a first aspect of the invention there is provided a method of controlling a spatial location of charged molecules in a reaction unit having a central electrode surrounded by a plurality of outer electrodes on a substrate, wherein the unit being filled with medium for providing frictional resistance to the motion of the molecules, the method comprising steps of: placing a charged molecule in said reaction unit; applying predetermined magnitudes of the first voltages to the central electrode and a set of at least 1 outer electrode selected from the plurality of outer electrodes to generate an electric field; and applying predetermined magnitudes of the second voltages to the central electrode and a set of at least 1 outer electrode selected from the plurality of outer electrodes to rotate the electric field for controlling the spatial location of the charged molecules, wherein the net charge, generated by the voltage applied to the selected electrodes, across the unit is maintained at zero.

According to a second aspect of the invention there is provided a method of controlling a spatial location of charged molecules in a reaction unit having a plurality of electrodes on a substrate, wherein the unit being filled with medium for providing frictional resistance to the motion of the molecules, the method comprising steps of: placing a charged molecule in said reaction unit; applying predetermined magnitudes of the first voltages to a set of at least 2 electrodes selected from the plurality of electrodes to generate an electric field; and applying predetermined magnitudes of the second voltages to a set of at least 2 other electrodes selected from the plurality of electrodes to rotate the electric field for controlling the spatial location of the charged molecules, wherein the net charge, generated by the voltage applied to the selected electrodes, across the unit is maintained at zero.

According to a third aspect of the invention there is provided a device for controlling a spatial location of charged molecules, comprising at least one reaction unit, having a substrate; a central electrode mounted on the substrate; a plurality of outer electrodes on the substrate, wherein the outer electrodes surround the central electrode; and medium for providing a frictional resistance to the charged molecule, wherein the medium covers the central electrode and outer electrodes on the substrate, whereby the location of the molecules is controlled by rotating an electric field generated by varying magnitudes of voltages applied to the electrodes, wherein the net charge, generated by the voltages applied to the selected electrodes, across the unit is maintained at zero.

According to a fourth aspect of the invention there is provided a device for controlling a spatial location of charged molecules, comprising at least one reaction unit, having a substrate; a plurality of electrodes on the substrate; and medium for providing a frictional resistance to the charged molecule, wherein the medium covers the electrodes on the substrate; whereby the location of the molecules is controlled by rotating an electric field generated by varying magnitudes of voltages applied to the electrodes, wherein the net charge, generated by the voltages applied to the selected electrodes, across the unit is maintained at zero.

According to a fifth aspect of the invention there is provided a device for reactions between molecules, comprising: a reaction unit having a substrate, a central electrode on the substrate, and a plurality of outer electrodes surrounding the central electrode on the substrate, wherein said central electrode is separated by a distance between one and 100 microns from said outer electrodes, and permeation layers with binding entities for reaction are placed on said central electrode; and a grounded outer ring electrode that surrounds said reaction unit to reduce the spread of the electric field generated by voltage applied to said electrodes over said reaction unit.

### BRIEF DESCRIPTION OF THE DRAWINGS

The above objects and advantages of the present invention will become more apparent by describing in detail preferred embodiments thereof with reference to the attached drawings in which:

FIG. 1 is the geometry of electrode placement in a reaction unit;

FIG. 2 is the pattern of electric application and rotation of electric field;

FIG. 3 is a clockwise fashion movement of electric field;

FIG. 4 is a molecular trajectory in moving electric field;

FIG. 5 is a reaction unit with an outer electrode ring;

FIG. 6A is a schematic diagram of electric field without outer-ring, and FIG. 6B is a schematic diagram of electric field with outer-ring;

FIG. 7 is a cross-section of a reaction unit fabricated using microlithography;

FIG. 8 is a matrix type device containing 50 reaction units;

FIG. 9 is the schematic diagram of a reaction unit structure;

FIG. 10A is the process of forming binding layer 1, and FIG. 10B is the process of forming binding layer 11; and

FIG. 11 is the schematic diagram of micro-machined device.

### DETAILED DESCRIPTION OF THE INVENTION

The concepts and embodiments of this invention are described in two sections. The first section is the method for controlling the spatial location of molecules, and the second is the design and fabrication of the chip for this method.

### DEFINITION OF THE TERM USED IN THIS INVENTION

The term “negative dominant bias” indicates that the polarity of the central electrode is set to be negative. In this invention the net charge of all electrodes is maintained at or near zero.

The term “positive dominant bias” indicates that the polarity of the central electrode is set to be positive. In this invention the net charge of all electrodes is maintained at or near zero.

The term “nucleic acid hybridization” is meant to include all hybridization reactions between all natural and synthetic forms and derivatives of nucleic acids, including: deoxyribonucleic acid (DNA), ribonucleic acid (RNA), polynucleotides and oligonucleotides.

The term “Polymerase Chain Reaction (PCR)” refers to a method of amplifying DNA fragments for analysis. PCR is composed of cycles of heating and cooling. A cycle begins with denaturing the DNA by heating (breaking apart the double strands of the DNA molecule into single-stranded DNAs). Then, primers (short pieces of DNA complementary to a specific sequence) bind to their specific part of every single-stranded DNA during cooling. This step is called annealing. In the final step, extension, the temperature is raised again and a specific enzyme called Taq polymerase is used to add complementary bases to the DNA from the end of the primer along the rest of the single-stranded DNA templates, thereby making multiple copies of the DNA segment between the two primers. The entire process is repeated multiple times (cycles) to yield a large amount of the desired DNA region. Since under ideal conditions the amount of DNA doubles with each cycle, 30 PCR cycles leads to a 2³⁰ (approximately a billion fold) increase in the amount of DNA as compared to the original sample.

The term “Self-Assembled Monolayers (SAMs)” refers to the ordered molecular assemblies that are formed spontaneously by the adsorption of a surfactant with a specific affinity of its headgroup to a substrate

The term “trapping” refers to the restriction of the spatial location of charged molecules (positive or negative) by the presence of an externally applied electric field. This restriction of spatial location prevents the charged molecules from entering or exiting the specified area while the electric field is in place.

### Section I. Method to Control the Spatial Location of Charged Molecules

Manipulation of two oppositely charged molecules, negative and positive, at the same time can provide many great advantages in biological analysis because most biological reactions require interactions between negatively charged molecules and positively charged molecules.

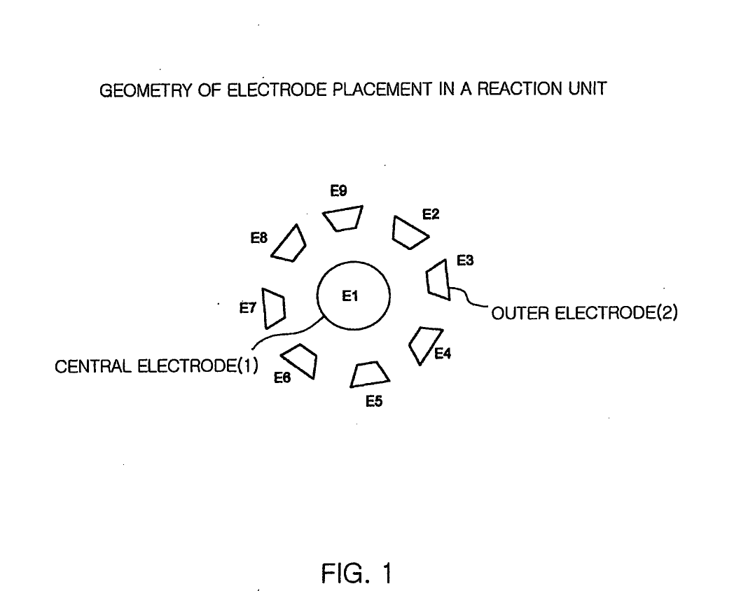

We realized a method to control spatial location of two oppositely charged molecules in a reaction unit at the same time. A special geometry of electrodes placement in a reaction unit makes it possible to manipulate two oppositely charged molecules and to position two charged molecules into a specific zone wherein desirable biological reactions occur. The geometry for this invention comprises a single central electrode surrounded by 3 or more outer electrodes in said reaction unit. FIG. 1 shows a typical geometry comprising single central electrode (1) surrounded by 8 outer electrodes (2) in a reaction unit as one preferred embodiment. At first, predetermined magnitudes of the first voltages are applied to a set of at least 2 electrodes selected from the plurality of electrodes to generate an electric field, wherein the net charge across the electrode of the unit is maintained at or near zero. The applied voltages may be positive or negative, being oriented in a manner that depends on the charge of the molecule to be trapped. And then, predetermined magnitudes of the second voltages are applied to a set of at least 2 outer electrodes selected from the plurality of outer electrodes to rotate the electric field for controlling the spatial location of the charged molecules. This application of voltage to the electrodes can be continued for trapping the charged molecule. The rotation can be in a clockwise or counter-clockwise fashion and performed by an orderly progression of electrode voltage wherein the voltage at a given electrode is set equal to the value at an adjacent electrode during the preceding time interval.

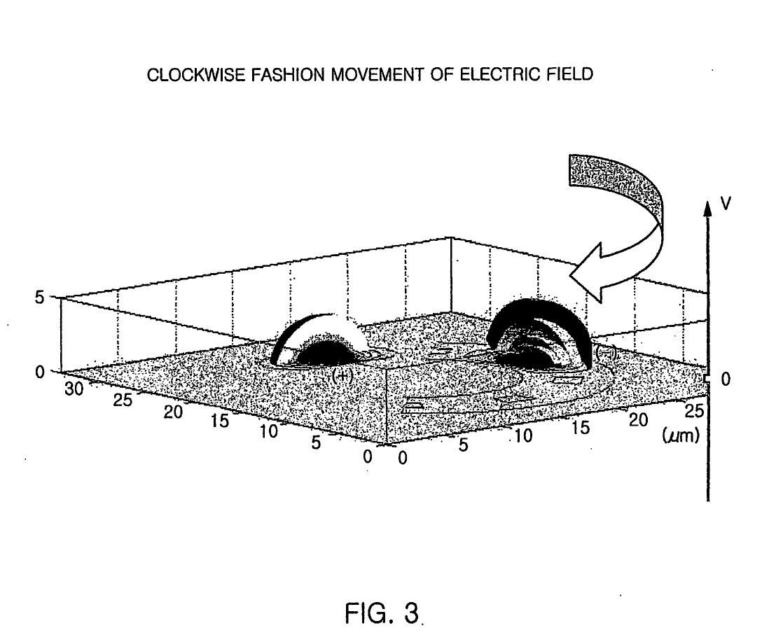

FIG. 3 shows an example of rotation of an electric field generated by applying positive and negative voltage to two outer electrodes respectively.

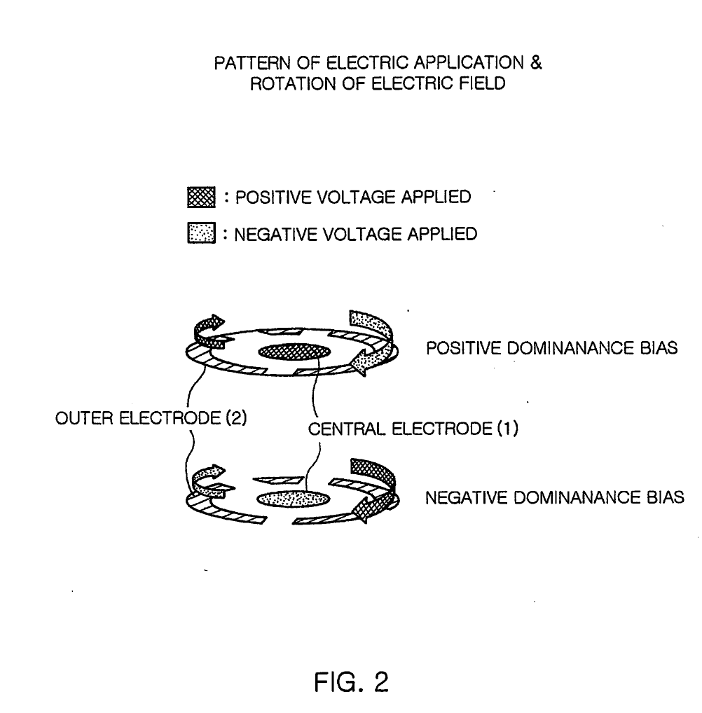

In this method, the voltages can also be applied to the central electrode to assist in the trapping the charged molecules. Application of voltage to these electrodes, maintaining the net charge at or near zero, can include a positive or negative dominant bias at the central electrode. FIG. 2 shows the pattern of electric field generated by applying voltage to the electrodes. To control the molecular trajectory, alternating positive dominant bias and negative dominant bias may be required. Also the electric field created can be configured so as to create an electronic trap from which charged molecules can not enter trap, and amplitude and rotational frequency of the electric field can be modulated in such a way as to separate ionic species of different electrophoretic mobility. While in one preferred embodiment, the central electrode is set to 0.1 V, an outer electrode is set to −2 V, and another outer electrode is set to 1 V, there are many possible combinations to set voltage to each electrode. Rotational frequency also affects the spatial location of charged molecules. For example, the rotational frequency should be larger, or the outer electrodes should be closer to the central electrode at lower voltage.

1. Parameters for Controlling of the Spatial Location of Molecules

1) Electric Charge:

To maintain the net charge of the electrodes at zero over a reaction unit, it is very important to set an appropriate value of voltage to each electrode. For a round electrode with a radius R and an applied voltage V, the total charge Q on the electrode is given by the equation: Q=8×V×R×.×. This assumes the charge distribution on the surface of the electrode is given by σ=Q/(2×.×R×SQRT(R²−ρ²)), where ρ is the distance from the center of the electrode and is the permittivity of free space. Clearly electrodes of different radii, but held at the same voltage, will have different amounts of electrical charge. According to this equation, when the central electrode is placed at a specified voltage, one or more outer electrodes would need to be at an opposite voltage to balance the charge. Due to the difference in radii of the central and outer electrodes, the balancing voltage will generally need to be several times higher than the voltage of the central electrode. In one preferred embodiment, the magnitude of voltage at the central electrode ranges from 0.01 to 01V, although this value is highly dependent on other factors.

For square or other electrodes geometries, the equation that yields the total charge will be slightly different. However, the charge will still depend on the size of the electrode. In addition to this, the presence of electric fields due to other electrodes in the system will alter the charge distribution on the surface of the electrodes. This makes the assumption related to σ less accurate as the externally applied field at the surface of an electrode becomes less uniform. However, this does not change the dependence of the total charge on electrode size.

2) Outer Ring:

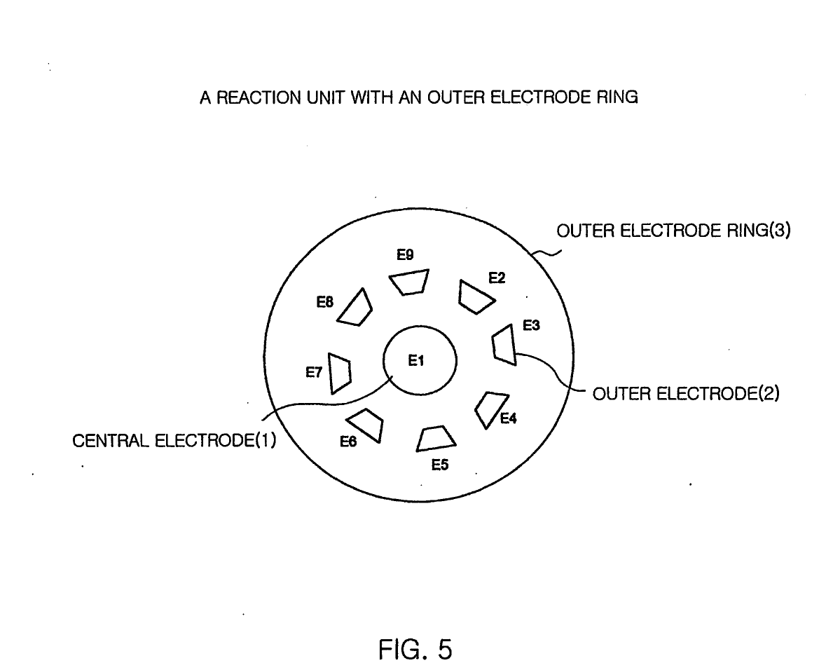

The electric field from a given reaction unit spreads in such a way that can interfere with the electric field at any adjacent reaction units. The inclusion of an outer ring electrode is required to reduce the effect of this spreading of the electric field. The outer ring electrode, when grounded, helps prevent the unwanted spread of a reaction unit's electric field. Mathematically, the grounded outer ring represents an additional boundary condition for the solution of Laplace's equation, the fundamental differential equation governing the behavior of electric fields in this case. By placing this additional boundary, the electrical potential, and hence the electric field, are forced to go to zero at the location of the ring electrode. This effect also extends vertically above the surface of the electrode. This does not force the potential to become zero at all points above the electrode, but does serve to reduce the potential. FIG. 5 shows a schematic diagram of a reaction unit surrounded by an outer electrode ring (3).

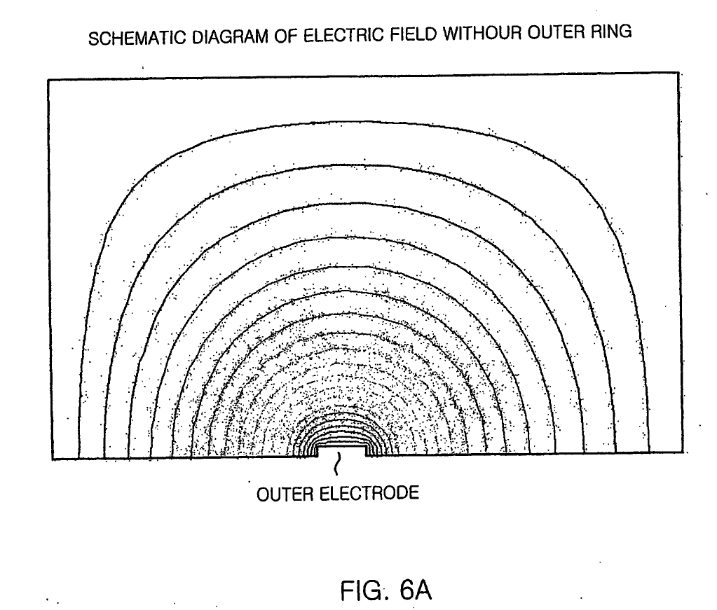

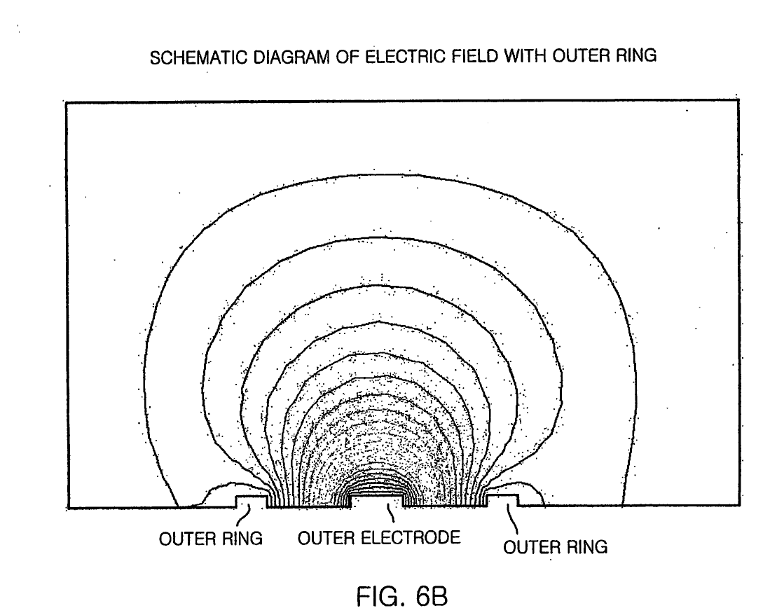

FIG. 6A and 6B illustrate the affect of the outer ring. FIG. 6A shows a schematic diagram of an electric field without a grounded outer ring and FIG. 6B shows a schematic diagram of the electric field with an outer ring. The presence of the outer electrode ring clearly reduces the spreading of the electric potential, minimizing the interference with adjacent units. In this case, independent and simultaneous control of the spatial location of charged molecules across multiple reaction units can be achieved. The array of this reaction unit can be used for high throughput screening or analysis of biological events such as DNA hybridization or polymerase chain reaction.

3) Electrodes:

For fine adjustment of the electric field allowing desirable biological reactions to occur, the number of electrodes should be optimized. The number of electrodes, depending on the reaction desired, can preferably range from 3 to 30. However, the number of electrodes has no limit since point electrodes can be used for the same invention. In one preferred embodiment, a single central electrode is surrounded by 8 outer electrodes. Inner electrodes within, yet electrically insulated from, the central electrode may also be needed for fine adjustment of the electric field for biological reactions. These inner electrodes can provide a way to control the spatial location of molecules more accurately for desired biological reactions.

4) Rotational Frequency:

The electric field can be rotated by alternating the application of voltage to a set of electrodes since each electrode can be independently controlled using a computer program. The rotational frequency may range from 0.001 Hz to 1,000 Hz, depending on the magnitude of voltage applied to the electrodes. The rotational frequency must be low enough to allow the molecules to keep up with the electric field as it changes. In one preferred embodiment, the frequency range from 0.01 Hz to 10 Hz. The rotational velocity of molecules is directly proportional to the electric field strength so that the time it takes for the molecules to move from one electrode to the next depends on the applied voltages as well as the distance between the electrodes. When the electric field is weaker, the rotation must be slower. At low electrode voltages the rotational frequency should be lower, or the outer electrodes should be closer together. Such a change in geometry may require more outer electrodes or a smaller central electrode.

In one preferred embodiment, the dimension of each electrode and the number of electrode are summarized at the following table 1.

TABLE 1
Dimensions of electrodes

| | |
|---|---|
| Central electrode radius | 100. |
| Inner/outer electrodes | 10. |
| Radius to center of outer electrodes | 130. |
| Radius to center of inner electrodes | 50. |
| Radius to outer ring | 200. |
| Width of outer ring | 20. |
| Distance between the center of two | 1,000˜2,000. |
| adjacent reaction units | |

TABLE 2
Value of each parameter in a reaction unit

| | |
|---|---|
| Minimum number of outer electrodes | 3 |
| Maximum number of outer electrodes | 30 |
| Minimum number of inner electrodes | 0 |
| Maximum number of inner electrodes | 8 |

| | | |
|---|---|---|
| Maximum voltage of any electrode | ±10 | volt |
| Minimum voltage for stimulation | 0.01 | volt |
| Minimum rotational frequency | 0.01 | Hz |
| Maximum rotational frequency | 10 | Hz |

5) Geometry

The geometry of electrodes placement in a reaction unit is one of key factors in this invention. For the goal of this invention, the placement of electrodes should create a closed system such as circular, square, or diamond-shape. In one preferred embodiment, the geometry is a toroidal form.

6) Others

Ion concentration—The ion concentration of buffer solution may also affect the mobility of molecules through the interaction with electric field generated by electrodes.

Probe—Some probes such as DNA molecules or proteins have an intrinsic electric charge, negative or positive. This may affect the electric field, though the effect is small when compared to the externally applied fields.

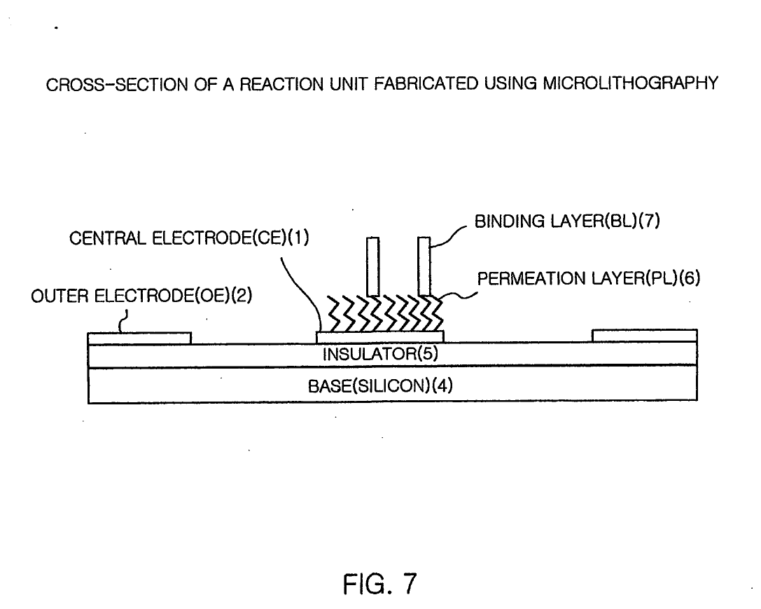

In addition to the invention, for biological reactions, any probe molecule including but not limited to DNA fragment, antibody, and enzymes can be anchored to the central electrode. FIG. 7 shows the cross-section of a reaction unit including the electrodes wherein probe should be anchored. A great number of attachment methods of probe to the electrodes have been studied, which vary widely in chemical mechanism, ease of use, probe surface density and attachment ability. (Farah N. Rehman, et al. Nucleic Acid Research, 1999, vol. 27, No. 2, p 649-655). Among the most promising solid phase attachment methods for this invention are those that utilize polyacrylamide supports and self-assembled monolayers (SAMs). Both may have a thiol group (—SH) that can be bound to the electrode or probe. The chief advantages of these supports are high probe capacity, low non-specific binding levels or relatively high thermal stability. Moreover, it is relatively easy to manipulate probe density for normalizing hybridization properties of a probe array. The attachment layer of polyacrylamide or SAMs can act as a permeation layer to allow the diffusion of gas generated by electrolysis. The permeation layer should have a pore limit property that inhibits or impedes the large binding entities, reactants, and analytes from physically contacting with the electrode. The permeation layer should also keep the active electrode surface physically from binding entities.

Once, the permeation layer is formed, alternation of positive and negative dominant bias leads charged molecules into high local concentrations around the central electrode to facilitate desired biological reactions.

### Section II. Design and Fabrication of a Device for Trapping Molecules

1. Overview

A device can be designed to have as few as one reaction unit or as many as hundreds or thousands of reaction units. In general, a complex device with a large number of reaction units is fabricated using microlithography techniques. Fabrication is carried out on silicon or other suitable substrate materials, such as glass, silicon dioxide, plastics, or ceramics. These microelectronic chip designs would be considered large-scale array or multiplex analysis devices. A device with a small number of reaction units or macro-scale reaction units would be fabricated using micro-machining techniques.

A reaction unit can be of any shape, preferably round, square, or rectangular. The size of a reaction unit depends on the size of electrodes placed said reaction unit. To make a reaction unit smaller than the resolution of microlithographic methods would require techniques such as electron beam lithography, ion beam lithography, or molecular beam epitaxy.

While a small reaction unit is desirable for analytical and diagnostic type applications, a larger reaction unit is desirable for applications such as, but not limited to, preparative scale biopolymer synthesis, sample preparation, electronically dispensing of reagents.

The electrode can be of any size, preferably range from sub-micron to several centimeters, with 5 micron to 100 micron being the most preferred size range for devices fabricated using microlithographic techniques, and 100 micron to 10 millimeters being the most preferred size range for devices fabricated using the micro-machining techniques. An electrode can be of any shape, preferably round or square.

The geometry of electrode placement in said reaction unit can be of any form, preferably round, square, or rectangular. In a preferred embodiment, one electrode is centered and is surrounded by the remaining electrodes.

The number of electrodes placed in said reaction unit can be more than three, preferably 3 to 30 electrodes. (In page 13, we said “The number of electrodes, depending on the reaction desired, can preferably range from 3 to 30. This exactly matches with the table 2.)

After a reaction unit, including electrodes, has been created by using microlithographic and/or micro-machining techniques, chemical modification, polymerization, or even further microlithographic fabrication techniques are used to create the specialized attachment and permeation layers. These important layers separate the binding entities from the metal surface of the electrode. This separation allows the DC mode wherein each electrode under the surface of a reaction unit can: (1) affect or cause the free field electrophoretic transport of molecules into a reaction zone wherein a reaction can occur; (2) concentrate molecules into a reaction zone wherein a reaction can occur; (3) continue to actively function in the DC mode after local concentration of molecules into a reaction zone; and (4) not adversely affect any biological reaction with electrochemical reactions and products.

1. Design Parameters (Microlithography)

Reaction Unit Fabrication:

As one preferred embodiment of the invention, FIG. 1 shows a basic geometry of electrode placement in a reaction unit fabricated using microlithography. Nine electrodes (E1˜E9) are formed by deposition of gold on an insulator matrix. One electrode is centered and is surrounded by the remaining electrodes (outer electrodes). The number of outer electrode is more than 3 and preferably 3-30. An insulator matrix separates the electrodes from each other. Insulators materials include, but are not limited to, silicon dioxide, silicone nitride, glass, resist, polyimide, rubber, plastic, or ceramic materials.

FIG. 7 shows the basic feature of a reaction unit fabricated microlithographically. The central electrode (CE) (1) and outer electrodes (OEs) (2) are formed on the matrix (base (4) and insulator (5)), and a permeation layer (PL) (6) and binding layer (BL) (7) is incorporated on the CE.

Thiols such as alkanethiols such as OH—((CH)₂)₆₋₁₀—SH in the permeation layer provide a base to which a probe is anchored. The permeation layer provides spacing between the electrode (CE) and the binding layer (BL) and allows solvent molecules, small counter-ions, and electrolysis reaction gases to freely pass to and from the electrode. It is possible to include within the permeation layer substances which can reduce the adverse physical and chemical effects of electrolysis reactions, including, but not limited to, redox reaction trapping substances, such as palladium for H₂, and iron complexes for O₂ and peroxides. The thickness of the permeation layer for microlithographically-produced devices can range from approximately 1 nanometers (nm) to 100 microns (.), with 2 nm to 10 μm being the most preferred.

The spacing between electrodes is determined by the ease of fabrication, the requirement for detector resolutions between electrodes, the nature of the electric field required for trapping, and the number of electrodes desired on a device. Particular spacing between electrodes is necessary for device function. This is because complex electric field patterns or dielectric boundaries are required to selectively move, separate, hold, or orient specific molecules in the space or medium between any of the electrodes. The device accomplishes this by controlling the spatial location of molecules in a limited zone around the probe anchored to thiols in the binding layer. Free field electrophoretic force provides for the rapid and direct transport of any charged molecules between any and all spatial locations on the device, or from the bulk solution to any electrode.

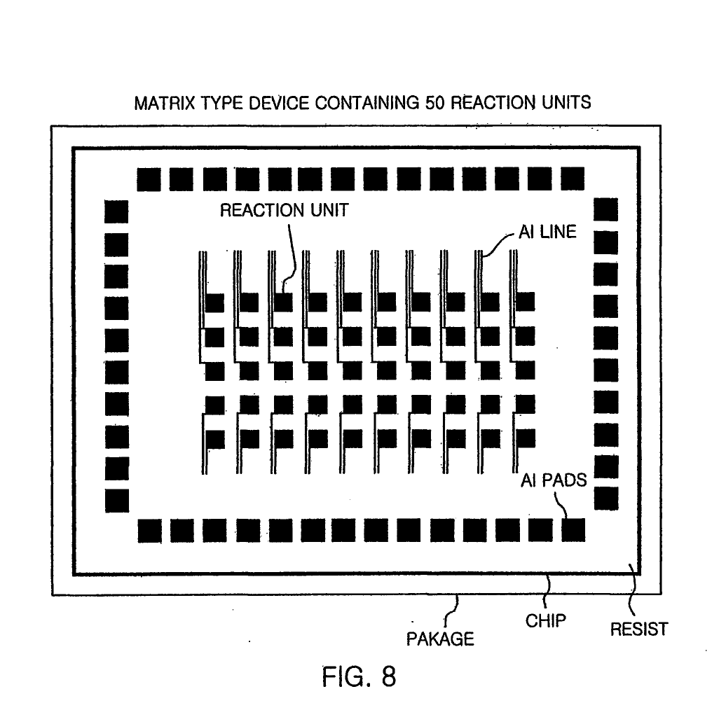

FIG. 8 shows a matrix type device containing 50 reaction units. A 50-reaction-unit device is a convenient design, which fits with standard microelectronic chip packaging components. Such a device is fabricated on a silicon chip substrate approximately 1.5 cm×1.5 cm, with a central area approximately 750×. 750 . containing the 50 reaction units. The number of reaction units on a matrix can range from 10 to 10,000, depending on an application as described above (thus, the dimension of electrodes placed on a reaction unit also vary in response to the application of this device.).

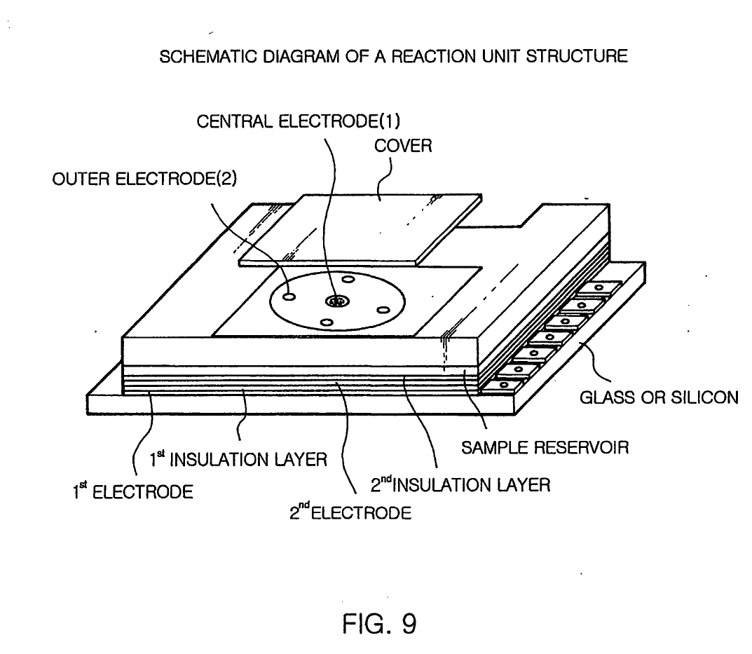

FIG. 9 shows a schematic diagram of the structure of a reaction unit that will be deposited on the matrix shown in FIG. 8.

Permeation and Binding Layer Formation

After fabrication, the central electrode on the device is modified with a specialized permeation and binding layer. This is an important aspect of the invention. The objective is to create on the electrode an intermediate permeation layer with selective diffusion properties and attachment surface layer with optimal binding properties.

The binding layer provides a base for the binding of various target probes such as DNA primer. The thickness of the binding layer for microlithographically-produced devices can range from 0.5 nm to 5., with 1 nm to 500 nm being the most preferred. Ideally, the specific binding layer is composed of (1) a self-assembled monolayer (SAM) or (2) polyacrylamide gel. Both of SAM or the gel provide spacing for the binding of large molecules on the central electrode of the reaction unit.

(1) SAM

Alkanethiol SAM is provided by the sulfur affinity for gold, and a comparably strong lateral interaction arising from the van der Waals forces between the chains. This lateral interaction can be controlled by changing the length of the hydrocarbon chain. The gold has the crystal structure of (111) face.

Optimally, the binding layer has from 10⁵ to 10⁷ functionalized locations per square micron (.²) for the attachment of specific binding entities. The attachments of binding entities should not overcoat or insulate the surface so as to prevent the underlying micro-electrode from functioning. A functional device requires some fraction (˜5% to 25%) of the actual metal micro-electrode surface to remain accessible to solvent (H₂O) molecules, and to allow the diffusion of counter-ions (e.g. Na⁺ and Cl⁻) and electrolysis gases (e.g., O₂ and H₂) to occur.

The intermediate permeation layer is also designed to allow diffusion to occur. Additionally, the permeation layer should have a pore limit property that inhibits or impedes the large binding entities, reactants, and analytes from physically contacting with the micro-electrode surface. The permeation layer keeps the active micro-electrode surface physically distinct from the binding layer of the electrode.

This design allows the electrolysis reactions required for electrophoretic transport to occur on micro-electrode surface, but avoids adverse electrochemical effects to the binding entities, reactants, and analytes.

The permeation layer can also be designed to include substances that scavenge adverse materials produced in the electrolysis reactions (H₂, O₂, free radicals, and etc.). A sub-layer of the permeation layer may be designed for this purpose.

A variety of designs and techniques can be used to produce the permeation layer. The general designs include: (1) physical adsorption, (2) chemical modification, (3) genetic engineering modification.

The arrangement of linear alkanethiol molecules in a vertical direction from the metal electrode surface can be formed by attaching liner alkanethiols directly to the metal electrode surface, with minimum cross linkage between the vertical structures. Ideally these molecules are bifunctional, with one terminal end suited for covalent attachment to the metal electrode surface, and the other terminal end suited for covalent attachment of binding entities.

Physical adsorption involves the direct immobilization of the probe molecule under investigation onto an unmodified substrate surface through hydrophobic or electrostatic interactions. Such processes are generally simple and involve no manipulation of the probe molecule. However, the direct immobilization of probe molecules onto a metal substrate has several disadvantages: some proteins are denatured and are subsequently inactivated upon direct contact with the metal surface; the binding between the protein and the metal surface is not stable; unspecific, random and multi-oriented immobilization of the protein onto the metal substrate causes of irreproducibility of results may occur; deposition of multi-layer may present problems; and the random orientation of the active site of the protein may prevent analyte binding.

An alternative to physical adsorption is offered by chemical modification of the probe. There are a wide variety of chemical manipulation procedures that result in the formation of a covalent bond between the protein and the substrate surface under non-denaturing conditions. In comparison with the results obtained using the direct immobilization method, chemical modification to produce a self-assembled biolayer offers a number of important advantages: reproducibility and stability of the protein monolayer on the substrate even upon exposure to a wide range of conditions; the possibility of controlling the density and environment of the immobilized species; the generation of uniform structures; higher coverage of the substrate surface; and the reduction of the number of possible random orientations that the protein can assume on the surface.

One preferred procedure that produces chemical modification-based monolayers involves the sulfur-containing compounds. The strong affinity of sulfur-containing compounds (thiols, thioethers and disulfides) for gold and other noble metals means that they can be excellent tagging molecules for protein modification and subsequent self-assembly, providing that a second functional group, that is able to form a covalent bond with the biomolecular, is present.

Two different approaches are utilized for chemical immobilization of probe: (1) Protein modification, (2) Substrate modification.

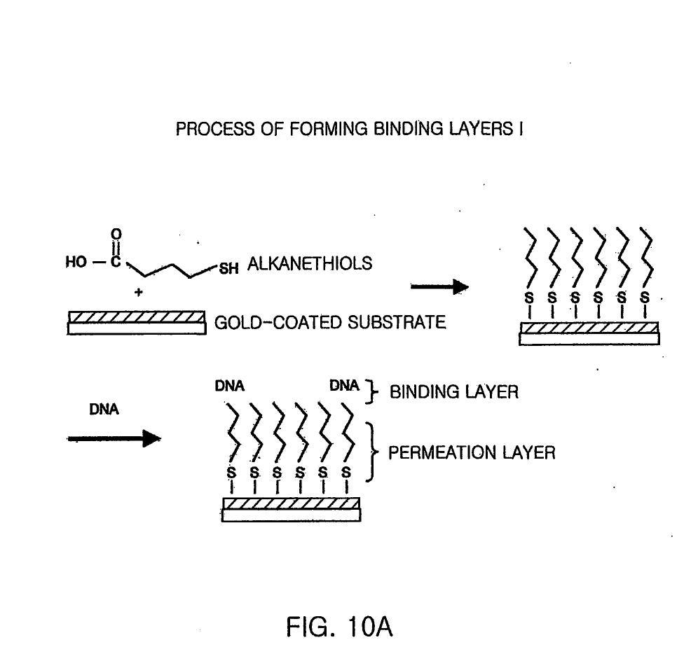

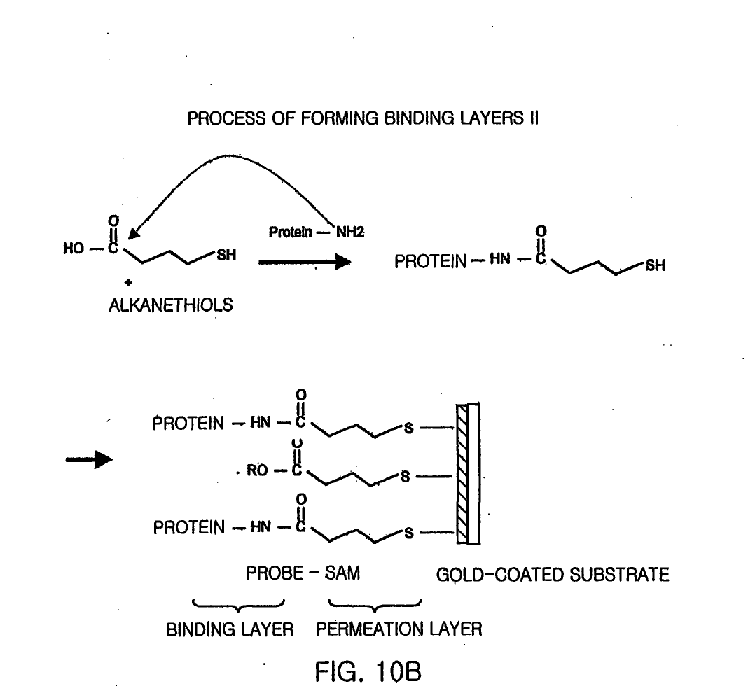

With protein modification, the protein of interest is tagged in solution with an appropriate sulfur-containing molecule via reaction with specific reactive groups present on the biomolecular surface, e.g. the . amino group of exposed lysine residues. The modified protein is then self-assembled onto a gold-coated substrate via the sulfur moiety. Substrate modification relies firstly on the modification of the substrate by self-assembling the sulfur-containing molecule. The protein is subsequently bound to the newly formed SAM via reaction with specific moieties present on the biomolecule surface. FIG. 10A shows a schematic process of alkanethiols containing DNA binding to the electrode surface. FIG. 10B shows the process of formation of protein binding layer. Protein reacts with alkanethiols first, and then the alkanethiols containing protein are bound to the electrode surface.

The third alternative for the immobilization of proteins and enzymes onto gold-coated substrates is offered by the recent availability of genetic engineering techniques. The use of genetic manipulation provides an opportunity to create unique attachment sites on protein surfaces and allows the formation of much more regular SAMs of proteins. Using genetic engineering techniques, it is possible to overcome the problem of multiple and random orientation of proteins on gold, a problem which is potentially still present with chemical modification. Enzymes can be immobilized in a fully active form after application of site-directed mutagenesis for the introduction of attachment sites in unique positions. The surface coverage of an engineered dihydrofolate reductase mutant, which lacked both its natural cysteines, on a gold-coated substrate is 4-fold lower than that obtained with a similar mutant that has one cysteine residue added directly to the C-terminus.

In order to obtain high quality monolayer structures, it is important to allow the full period of time for SAM formation. Following deposition of the monolayer, the surface is exposed to extensive buffer washing to remove any protein material not self-assembled at the surface, which might have been deposited as a multi-layer.

(2) Polyacrylamide Gel

Polyacrylamide gel can be used as a base for the binding of large molecules on the central electrode of the reaction unit. For example, polyacrylamide gel having a high density of hybridizable oligonucleotide can be produced with acrylamide modifications. Oligonucleotide bearing 5′-terminal acrylamide modifications can efficiently copolymerize with acrylamide monomers to form thermally stable DNA-containing polyacrylamide co-polymers. (Farah N. Rehman, et al. Nucleic Acid Research, 1999, vol. 27, No. 2, p 649-655). Because this invention does not cover this modification, and there are many products commercially available for the gel for this invention, for example, Mosaic Technologies, Inc., further discussion stops here.

Micro-Machined Device Design and Fabrication

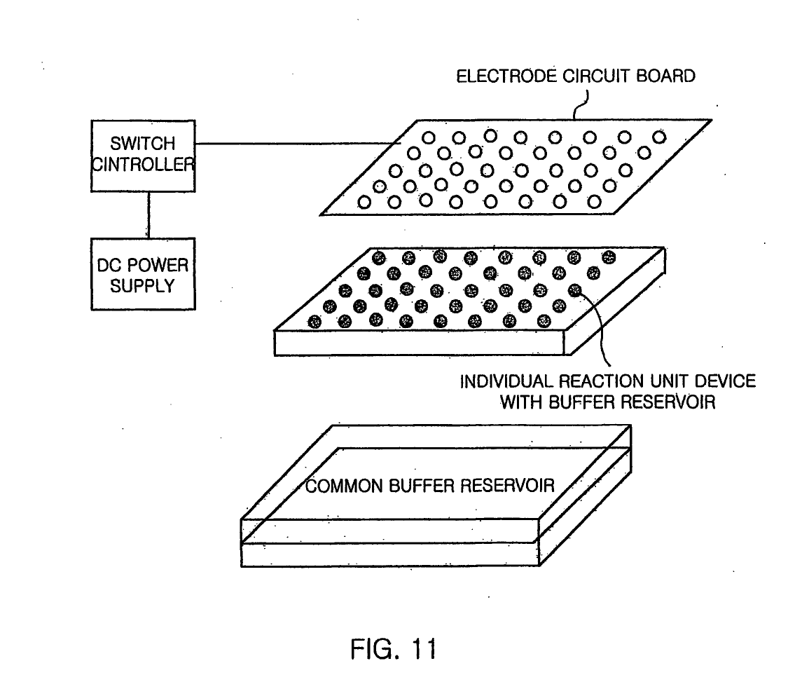

This section describes how to use micro-machining techniques (e.g., drilling, milling, etc.) or non-lithographic techniques to fabricate devices. FIG. 11 shows the schematic diagram of micro-machined device. In general, these devices have relatively larger micro-electrodes (>100 microns) than those produced by microlithography. This device can be used for analytical applications, as well as for preparative type applications, such as biopolymer synthesis, sample preparation, and reagent dispensing. Storage locations can be fabricated in three-dimensional formats (e.g., tubes or cylinders) in order to carry large amounts of binding entities. Such devices can be fabricated using a variety of materials, including, but not limited to, plastic, rubber, silicon, glass (e.g., microchannelled, microcapillary, etc.), or ceramics. Low fluorescent materials are more ideal for analytical application. In the case of micro-machined devices, connective circuitry and large electrode structures can be printed onto materials using standard circuit board printing techniques known to those skilled in the art.

Once a device has been bound with specific binding entities, a variety of molecular biology type multi-step and multiplex reactions and analyses can be carried out on the device. The devices of this invention are able to electronically provide active and dynamic control over a number of important reaction parameters. This electronic control leads to new physical mechanisms for controlling reactions, and significant improvements in reaction rates, specificities, and sensitivities. The improvements in these parameters come from the ability of the device to electronically control and directly affect: (1) the rapid transport of reactants or analytes to a specific reaction unit containing attached specific binding entities; (2) an increase in reaction rate due to the concentration of reactants or analytes with the specific binding entities on the surface of the specific micro-electrode; (3) the rapid and selective removal of un-reacted and non-specifically bound components from the micro-electrode; (4) small amount of samples required for biological analysis since each reaction unit can use the sample in series. Each reaction can use the same sample as the previous reaction because the original sample is not modified by the reaction for certain reactions. So it is possible to conduct multiple analyses with small amounts of sample material since each reaction unit is independently controlled; (5) some components necessary for biological reactions can be anchored to the central electrode if necessary; and (6) the product(s) produced by a biological reaction can be removed from of the binding layer for subsequent reactions. For example, in PCR, cloned DNA can be detached from the binding layer and another extension can be done using the same bound primer sequence.

The self-addressed devices of this invention are able to rapidly carry out a variety of micro-formatted multi-step and/or multiplex reactions and procedures; which include, but are not limited to: DNA and RNA hybridizations procedures and analysis in conventional formats: e.g., attached target DNA/probe DNA, attached probe DNA/target DNA, attached capture DNA/target DNA/probe DNA; multiple or multiplexed hybridization reactions in both serial and parallel fashion; restriction fragment and general DNA/RNA fragment size analysis; molecular biology reactions, e.g., restriction enzyme reactions and analysis, ligase reactions, kinasing reactions, and DNA/RNA amplification; antibody/antigen reactions involving large or small antigens and haptens; diagnostic assays, e.g., hybridization analysis (including in-situ hybridization), gene analysis, fingerprinting, and immunodiagnostics; sample preparation, cell sorting, selection, and analysis; biomolecular conjugation procedure (i.e. the covalent and non-covalent labeling of nucleic acids, enzymes, proteins, or antibodies with reporter groups, including fluorescent, chemiluminescent, calorimetric, and radioisotopic labels); biopolymer synthesis, e.g., combinatorial synthesis of oligonucleotides or peptides; water soluble synthetic polymer synthesis, e.g., carbohydrates or linear polyacrylates; and macromolecular and nanostructure (nanometer size particles and structures) synthesis and fabrication.

Nucleic acid hybridization or PCR can be used as main examples of this invention because of their importance in diagnostics, and because they characterize one of the more difficult types of binding (affinity) reactions. This is particularly true when they are carried out in multiplex formats, where each individual hybridization reaction requires a different stringency condition.

The device and methods allow nucleic acid hybridization to be carried out in a variety of conventional and new formats. The ability of the device to electronically control reaction parameters greatly improves nucleic acid hybridization analysis, particularly the ability of the device to provide electronic stringency control (ESC) to each individual micro-electrode on an array. In essence, this allows each individual hybridization reaction on a common array to be carried out as a single test tube assay.

The term “nucleic acid hybridization” is meant to include all hybridization reactions between all natural and synthetic forms and derivatives of nucleic acids, including: deoxyribonucleic acid (DNA), ribonucleic acid (RNA), polynucleotides and oligonucleotides.

Conventional polymerase chain reaction (PCR) formats can be carried out with the disclosed device as well as large-scale array or matrix formats. As an example, an APEX (Active Programmable Electronic Matrix) device like NanoChip™ developed by Nanogen for DNA amplification and analysis is designed, fabricated, and used in the following manner. Arrays of reaction unit including microelectrodes are first fabricated using microlithographic (or micromachining) techniques. The number of reaction units and the geometry of electrodes placement on the reaction unit depends on the final use. The device is rapidly addressed in a serial manner with a mixture of alkanethiols including oligonucleotide as DNA primers. In this case, the oligonucleotides are 3′-terminal oligonucleotides in the range of 6-mers to 100-mers, larger polynucleotides can be attached if desired. The thiols functional group of alkanethiols allows for covalent attachment to the specific micro-electrode attachment surface (FIG. 10A). This group of specific oligonucleotides can be readily synthesized on a conventional DNA synthesizer using conventional techniques. The synthesis of each specific oligonucleotide is initiated from a ribonucleotide controlled pore glass (CPG) support. Thus, the 3′-terminal position contains a ribonucleotide, which is then easily converted after synthesis and purification to a terminal dialdehyde derivative by periodate oxidation. The hydroxy group of alkanethiols containing oligonucleotides will react readily with the primary amine functional groups on the surface of electrodes.

### EXAMPLE

This example illustrates the ability of this technology to control the spatial location of charged particles in a reaction chamber.

The reaction chamber consists of 10 independently-addressable gold electrodes embedded in an insulating material. The insulator and electrode surfaces are covered by a permeation layer and buffer solution commonly used in biological experiments.

The electrodes are arranged with a single, large, circular electrode in the center. The radius of the central electrode in this embodiment is 35 microns. The central electrode is surrounded by eight outer electrodes. These outer electrodes are also round, and are located uniformly about the central electrode. The radius of the outer electrodes is 5 microns. The distance between the middle of the central electrode and the middle of the outer electrodes is 45 microns. This leaves a gap of 5 microns between the conducting surfaces of the central and outer electrodes. With eight evenly-spaced outer electrodes, the angle between the outer electrodes is 45 degrees, relative to the middle of the central electrode. This results in a center-to-center distance of 34.4 microns between outer electrodes. The resulting gap between conducting areas of outer electrodes is thus 24.4 microns. The outer electrodes are surrounded by a ring of conducting material, the inner edge of which is located 70 microns from the middle of the central electrode. The ring is 10 microns wide and is always maintained at zero volts. The other electrodes' voltages vary depending on the operation being performed in the reaction unit.

In the simulations performed, the buffer was assumed to have the following properties. A viscosity equal to that of pure water, 0.891×10⁻³ kg/m×s, a temperature of 25° C., a dielectric constant equal to that of water, 78.3, a pH of 8.3, a total ionic strength of 0.256M. The particles were assumed to have a mobility of 3×10⁻⁴ cm²/(volt×sec).

To induce motion in the charged particles present in this buffer an electric field was produce by application of known voltages to the electrodes described above. The outer electrodes can be numbered 1 through 8, starting with the 12 o'clock position and moving clockwise. The voltage at electrode 1 can be set to a specific value, V1, electrode 2 set to V2, etc. The field thus created is said to rotate when the voltage at electrode 2 is set equal to V1, electrode 3 is set to V2, etc. Repeating this pattern of rotating the electric field results in a circular motion of nearby charged particles. This can be achieved by setting electrode 1 at −10 volts, electrode 4 at 10 volts, and all other electrodes to zero volts. In this configuration, negatively charged particles are attracted to electrode 4 while positively charged particles are preferentially attracted to electrode 1. As the field is rotated clockwise by systematically shifting the voltage at the two non-zero electrodes, charged particles are attracted to the new location of the electrode of opposite polarity. In this way the particles can be lead in a circle around the central electrode.

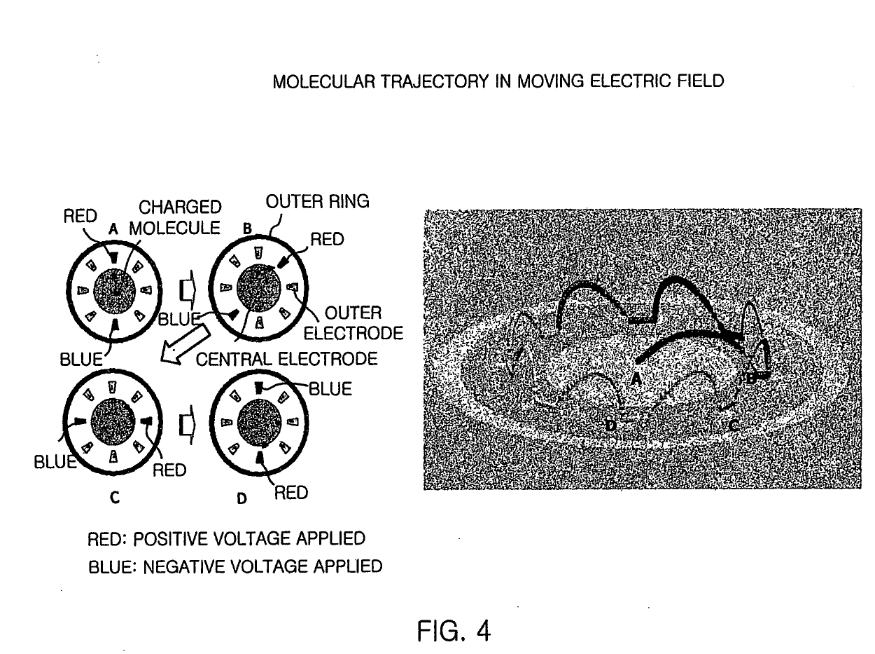

The voltages used in this rotation affect the pattern of motion of the particles. Increasing the voltage allows the electric field to extend farther into the volume beyond the reaction chamber, but the grounded ring helps prevent this spread along the surface of the chip. As the field extends further due to larger voltages, increasing the voltage can help attract charged particles from larger distances. This can be used to increase the local concentration of charged particles near the central electrode. Larger voltages also result in faster motion of the particles. The velocity of particles is equal to the product of their mobility and the electric field. Due to the geometry of the system the electric field lines are not straight, but rather create arcs that extend between electrodes. This arcing shape is also seen in the trajectory of charged particles. The height to which particles travel above the surface of the chip increases as the voltage is increased. FIG. 4 shows the trajectory caused by rotating voltages of −4, −1, −1, and 10 volts for electrodes 1, 2, 3, and 4 respectively. All other electrodes were grounded. Setting electrode 2 to a value of −1 volt causes any negative particles near that surface to be pushed away prior to the rotation of the field. This is beneficial as the −4 volt value would cause those same particles to be ejected more forcefully, and to greater heights. This may be undesirable for some applications, as it increases to total distance the particle must travel between electrodes. This increase in distance can affect the frequencies at which the rotation must be carried out. That is, if the field is to be rotated in such a way that particles can keep up with this rotation, care must be taken to account for their total path length. The voltage at the electrode trailing the 10 volt value must be at least slightly negative. This is because the large positive voltage attracts negative particles. If that value rotates away, and is replaced by zero, the particles near that surface are largely screened from the field of adjacent electrodes. Placing the electrode at a negative value pushes the negative particles away from the surface, allowing them to interact more strongly with the field from adjacent electrodes. Without this (as in the first example with only +/−10 volt electrodes), most particles attracted by either electrode will remain trapped near the surface of that electrode until the oppositely charged electrode rotates to that location. At this time the particles with be repelled strongly, and pushed far above the surface of the chip. This effect is also modulated by the thickness, density, and material properties of the permeation layer.

The geometry of the electrodes also affects the particle trajectory. The larger the distance from the middle of the central electrode to the middle of the outer electrodes, the lower the rotation frequency must be for particles of the same mobility. This is simply due to the increased path length between outer electrodes, and a corresponding decrease in the electric field produced by a given voltage at the two outer electrodes. This increased distance could be compensated for by increasing the voltage at the outer electrodes. Such an increase would allow the use of higher rotation frequencies, but would also result in a path with a larger arc. The relationship between electrode geometry, rotation frequency, and voltages is highly interrelated. Other considerations may place limits on one or more of these values, such as the sensitivity of biochemical processes to the externally applied electric field created by the electrodes. For example, in applications that cannot tolerate large fields, it is necessary to either reduce the radii of the electrodes or the frequency of rotation. With the geometry and voltages stated above, rotation frequencies of 1 Hz to 4 Hz result in trajectories identical to that shown in FIG. 4. Particles in such a trajectory are said to be trapped. They can be held indefinitely in this orbit until needed. Being located near the central electrode, where all targets are bound, the trapped particles may be made available for reactions very rapidly.

1. A method of controlling a spatial location of charged molecules in a reaction unit having a central electrode surrounded by a plurality of outer electrodes on a substrate, wherein the unit is filled with a medium for providing frictional resistance to motion of the molecules, the method comprising:
placing a charged molecule in the reaction unit;
applying predetermined magnitudes of first voltages to the central electrode and a set of at least one outer electrode selected from the plurality of outer electrodes to generate an electric field; and
applying predetermined magnitudes of second voltages to the central electrode and a set of at least one outer electrode selected from the plurality of outer electrodes to rotate the electric field for controlling the spatial location of the charged molecules, wherein net charge generated by the voltages applied to the electrodes selected, across the unit, is maintained at zero.

2. A method of controlling a spatial location of charged molecules in a reaction unit having a plurality of electrodes on a substrate, wherein the unit is filled with a medium for providing frictional resistance to motion of the molecules, the method comprising:
placing a charged molecule in the reaction unit;
applying predetermined magnitudes of first voltages to a set of at least two electrodes selected from the plurality of electrodes to generate an electric field; and
applying predetermined magnitudes of second voltages to a set of at least two other electrodes selected from the plurality of electrodes to rotate the electric field for controlling the spatial location of the charged molecules, wherein net charge generated by the voltages applied to the electrodes selected, across the unit, is maintained at zero.

3. The method of claim 1, wherein the number of outer electrodes is from three to thirty.

4. The method of claim 2, wherein the number of electrodes is from three to thirty.

5. The method of claim 1, including a probe molecule anchored to said central electrode.

6. The method of claim 1 including applying voltages to the electrodes in a range from −10V to +10V.

7. A method of claim 5, wherein the probe is selected from the group consisting of DNA, RNA, enzymes, and proteins.

8. The method of claim 1, wherein the central electrode includes at least one inner electrode within the central electrode, electrically insulated from the central electrode.

9. The method of claim 1, including rotating the electric field generated by the electrodes by applying an orderly progression of electrode voltages and setting the voltage at a given electrode at a present time interval equal to the voltage at an adjacent electrode at a preceding time interval.

10. The method of claim 1, including modulating amplitude and rotational frequency of the electric field to separate ionic species of different electrophoretic mobility.

11. The method of claim 1, wherein the electric field has a rotational frequency from 0.001 Hz to 1000 Hz.

12. The method of claim 1, including creating the electric field to produce an electronic trap which charged molecules cannot enter.

13. The method of claim 12, including temporarily re-configuring the electric field to allow specific charged molecules to enter the trap.

14. The method of claim 1, wherein the charged molecule is selected from the group consisting of DNA, RNA, proteins, and enzymes relating to biological reactions.

15. The method of claim 14, wherein the biological reactions include nucleic acid hybridizations, nucleic acid amplification, sample preparation, antibody/antigen reactions, clinical diagnostics, and biopolymer synthesis.

16. A method of claim 1, including changing temperature in the reaction unit incrementally.

17. The method of claim 1, including changing pH in the reaction unit incrementally.

18. The method of claim 1, including changing ion concentrations in the reaction unit incrementally.

19. The method of claim 1, wherein the medium is selected from the group consisting of a buffer solution and a porous material.

20. The method of claim 1, including a permeation layer and binding layer on the central electrode.

21. The method of claim 20, wherein said permeation layer is selected from the group consisting of self-assembled monolayers of alkanethiols and a porous material.

22. The method of claim 1, wherein the electrode is an electrically conductive material selected from the group consisting of gold, silver, copper, and aluminum.

23. The method of claim 1, wherein the substrate is an electrically insulating material selected from the group consisting of glass, silicon, and ceramics.

24. The method of claim 1, wherein the outer electrodes form a closed shape.

25. The method of claim 2, wherein the electrodes form a closed shape.

26. A device for controlling a spatial location of charged molecules, comprising:
at least one reaction unit having
a substrate;
a central electrode mounted on the substrate;
a plurality of outer electrodes on the substrate, wherein the outer electrodes surround the central electrode; and
a medium providing frictional resistance to movement of a charged molecule, wherein the medium covers the central electrode and the outer electrodes on the substrate, whereby location of the molecule is controlled by rotating an electric field generated by varying magnitudes of voltages applied to the central and outer electrodes, and net charge generated by the voltages applied to the selected electrodes, across the unit, is maintained at zero.

27. A device for controlling a spatial location of charged molecules, comprising:
at least one reaction unit having
a substrate;
a plurality of electrodes on the substrate; and
a medium for providing a frictional resistance to movement of a charged molecule, wherein the medium covers the electrodes on the substrate, whereby location of the molecule is controlled by rotating an electric field generated by varying magnitudes of voltages applied to the electrodes, and net charge generated by the voltages applied to the selected electrodes, across the unit, is maintained at zero.

28. The device of claim 26, further comprising an outer ring surrounding the outer electrodes on the substrate, wherein the outer ring reduces spreading over said reaction unit of the electric field generated by voltages applied to said electrodes.

29. The device of claim 26, wherein the number of electrodes is from three to thirty.

30. The device of claim 26, wherein the electrodes are located to form a closed shape, such as a circle, a rectangle, and a diamond-shape.

31. The device of claim 26, including a probe molecule anchored to said central electrode.

32. The device of claim 31, wherein said probe is selected from the group consisting of DNA, RNA, enzymes, and proteins.

33. The device of claim 26, wherein the central electrode includes at least one inner electrode within the central electrode, electrically insulated from the central electrode.

34. The device of claim 26, wherein the electric field generated by the electrodes is caused to rotate by applying an orderly progression of electrode voltages and the voltage at a given electrode at a present time interval is set equal to the voltage at an adjacent electrode at a preceding time interval.

35. The device of claim 26, wherein amplitude and rotational frequency of the electric field is modulated to separate ionic species of different electrophoretic mobility.

36. The device of claim 26, wherein the electric field is configured to create an electronic trap which charged molecules cannot enter.

37. The device of claim 36, wherein the electric field is temporarily re-configured to allow specific charged molecules to enter the trap.

38. The device of claim 26, wherein the charged molecule is selected from the group consisting of DNA, RNA, proteins and enzymes relate to biological reactions.

39. The device of claim 38, wherein the biological reactions include nucleic acid hybridizations, nucleic acid amplification, sample preparation, antibody/antigen reactions, clinical diagnostics, and biopolymer synthesis.

40. The device of claim 26, wherein temperature in the reaction unit is changed incrementally.

41. The device of claim 26, wherein pH in the reaction unit is changed incrementally.

42. The device of claim 26, wherein ion concentration in the reaction unit is changed incrementally.

43. The device of claim 26, wherein said medium is selected from the group consisting of a buffer solution and a porous material.

44. The device of claim 26, including a permeation layer and binding layer on said central electrode.

45. The device of claim 44, wherein said permeation layer is selected from the group consisting of self-assembled monolayers of alkanethiols and porous material.

46. The device of claim 26, wherein said electrode is an electrically conductive material selected from the group consisting of gold, silver, copper, and aluminum.

47. The device of claim 26, wherein said substrate is an electrically insulating material selected from the group consisting of glass, silicon, and ceramics:

48. The device of claim 26, wherein the outer electrodes form a closed shape.

49. The device of claim 27, wherein the electrodes form a closed shape.

50. A device for reactions between molecules, comprising:
a reaction unit having
a substrate,
a central electrode on the substrate,
a plurality of outer electrodes surrounding the central electrode on the substrate, wherein said central electrode is separated by a distance between one and one hundred microns from said outer electrodes, and permeation layers with binding entities for reaction are placed on said central electrode; and
a grounded outer ring electrode that surrounds said reaction unit, said outer ring electrode reducing spreading over said reaction unit of the electric field generated by voltage applied to said electrodes.

51. The method of claim 2 including applying voltages to the electrodes in a range from −10V to +10V.

52. The method of claim 2, including rotating the electric field generated by the electrodes by applying an orderly progression of electrode voltages and setting the voltage at a given electrode in a present time interval equal to the voltage at an adjacent electrode at a preceding time interval.

53. The method of claim 2, including modulating amplitude and rotational frequency of the electric field to separate ionic species of different electrophoretic mobility.

54. The method of claim 2, wherein the electric field has a rotational frequency from 0.001 Hz to 1000 Hz.

55. The method of claim 2, including creating the electric field to produce an electronic trap which charged molecules cannot enter.

56. The method of claim 55, including temporarily re-configuring the electric field to allow specific charged molecules to enter the trap.

57. The method of claim 2, wherein the charged molecule is selected from the group consisting of DNA, RNA, proteins, and enzymes relating to biological reactions.

58. The method of claim 57, wherein the biological reactions include nucleic acid hybridizations, nucleic acid amplification, sample preparation, antibody/antigen reactions, clinical diagnostics, and biopolymer synthesis.

59. A method of claim 2, including changing temperature in the reaction unit incrementally.

60. The method of claim 2, including changing pH in the reaction unit incrementally.

61. The method of claim 2, including changing ion concentrations in the reaction unit incrementally.

62. The method of claim 2, wherein the medium is selected from the group consisting of a buffer solution and a porous material.

63. The method of claim 2, wherein the electrode is an electrically conductive material selected from the group consisting of gold, silver, copper, and aluminum.

64. The method of claim 2, wherein the substrate is an electrically insulating material selected from the group consisting of glass, silicon, and ceramics.

65. The device of claim 27, further comprising an outer ring surrounding the electrodes on the substrate, wherein the outer ring reduces spreading over said reaction unit of the electric field generated by voltages applied to said electrodes.

66. The device of claim 27, wherein the number of electrodes is from three to thirty.

67. The device of claim 27, wherein the electrodes are located to form a closed shape, such as a circle, a rectangle, and a diamond-shape.

68. The device of claim 27, wherein the electric field generated by the electrodes is caused to rotate by applying an orderly progression of electrode voltages and the voltage at a given electrode in a present time interval is set equal to the voltage at an adjacent electrode at a preceding time interval.

69. The device of claim 27, wherein amplitude and rotational frequency of the electric field is modulated to separate ionic species of different electrophoretic mobility.

70. The device of claim 27, wherein the electric field is configured to create an electronic trap which charged molecules cannot enter.

71. The device of claim 36, wherein the electric field is temporarily re-configured to allow specific charged molecules to enter the trap.

72. The device of claim 27, wherein the charged molecule is selected from the group consisting of DNA, RNA, proteins, and enzymes relate to biological reactions.

73. The device of claim 38, wherein the biological reactions include nucleic acid hybridizations, nucleic acid amplification, sample preparation, antibody/antigen reactions, clinical diagnostics, and biopolymer synthesis.

74. The device of claim 27, wherein temperature in the reaction unit is changed incrementally.

75. The device of claim 27, wherein pH in the reaction unit is changed incrementally.

76. The device of claim 27, wherein ion concentration in the reaction unit is changed incrementally.

77. The device of claim 27, wherein said medium is selected from the group consisting of a buffer solution and a porous material.

78. The device of claim 27, wherein said electrode is an electrically conductive material selected from the group consisting of gold, silver, copper, and aluminum.

79. The device of claim 27, wherein said substrate is an electrically insulating material selected from the group consisting of glass, silicon, and ceramics.
